(19) United States
(12) Patent Application Publication
(10) Pub. No.: US 2001/0055776 A1
(43) Pub. Date: Dec. 27, 2001

(54) High throughput cell-based assay kits

(76) Inventor: Dale Greenwalt, Boyds, MD (US)

Correspondence Address: BANNER & WITCOFF, 1001 G STREET N W, SUITE 1100, WASHINGTON, DC, 20001, US

(21) Appl. No.: 09/882,023

(22) Filed: Jun. 18, 2001

Related U.S. Application Data
(62) Division of application No. 09/501,786, filed on Feb. 11, 2000.

(51) Int. Cl. G01N 33/53; G01N 33/567

(52) U.S. Cl. 435/7.1; 435/7.21

(57) ABSTRACT

A new assay is described that incorporates the time saving features of a lanthanide chelate-conjugated ligand and a filter plate on which cells can be both cultured and washed in situ with vacuum assist. Loss of cells during washing steps is significantly curtailed through the use of the cell-culture-compatible filter plates, and the high endogenous fluorescence backgrounds characteristic of cell-based assays is avoided by use of time-resolved fluorescence. The assay described herein permits the screening of large libraries of compounds and/or recombinant proteins for either toxicity or stimulation of differentiation.

### TECHNICAL AREA OF THE INVENTION

The invention relates to the detection of marker proteins in cell culture. More particularly, the invention relates to high throughput screening methods for agents which affect the expression of marker proteins and to the related test kits.

### BACKGROUND OF THE INVENTION

The relative paucity of high- or rapid-throughput cell-based assays is a significant bottleneck in the realization of proteonomics' full potential for drug discovery. For example, the most commonly used in vitro assay of the proliferative or differentiative response of hematopoietic cells is the colony assay (1). The colony assay utilizes hematopoietic progenitors from human bone marrow or umbilical cord blood in clonogenic assays in a semisolid medium. Colonies of differentiating blood cells appear over a time course of 10 to 14 or more days and are counted manually. While the parameters of this assay have been optimized to obtain accurate and quantitative data, the extremely low throughput of this assay hinders its use in library screening and drug discovery.

A number of cell-based assay formats are currently in widespread use. In the field of hematopoiesis, screening for novel factors that stimulate erythropoiesis may utilize cell-based assays of Ca⁺⁺ mobilization or membrane potential in primary hematopoietic cells or cell lines derived from various leukemias. Cell-based assays which measure a differentiative response require either the transfection of a target cell with a reporter gene tied to the expression of a differentiation-specific marker protein or an immunoassay for a differentiation-specific marker. Cell-based gene reporter assays of differentiation often rely on the use of primary cells which are difficult to transfect.

Immunoassays are further complicated by the need to remove unbound antibody. The rinse steps in a cell-based immunoassay are particularly time-consuming if nonadherent cells are used, because centrifuigation of the cells is required. When working with adherent cells, use of a plate washer risks loss of cells. Warren et al. (2) devised an innovative cell-based immunoassay in which cells were transferred to a fixative-treated assay plate and fixed in place prior to incubations with primary and secondary antibodies. While this assay has been used successfully, transfer of cells from the culture plate to the assay plate is time consuming and often is incomplete.

Thus, there is a need in the art for cell-based assays which are more rapid and efficient and which can be used to screen test compounds for toxicity or for the ability to affect differentiation of a progenitor cell.

### SUMMARY OF THE INVENTION

It is an object of the invention to provide methods of detecting a marker protein of a cell which can be used in a high throughput screening format. This and other objects of the invention are provided by one or more of the embodiments described below.

One embodiment of the invention is a method of detecting a marker protein of a cell. The cell is cultured on a filter plate which has a low background phosphorescence and pores which permit the cell to be cultured on the filter plate and to be washed in situ under vacuum assist. The cell is contacted with a reagent comprising a ligand which specifically binds to the marker protein and a lanthanide chelate. The lanthanide chelate comprises a lanthanide ion and a chelating agent. The chelating agent is bound to the ligand. The cell is washed to remove reagent which is not bound to the marker protein to provide a washed cell. The washed cell is observed for a sufficient time to detect fluorescence associated with the lanthanide ion which is at least two-fold greater relative to any background fluorescence. Detection of the fluorescence associated with the lanthanide ion indicates the presence of the marker protein. Optionally, the cell is cultured in the presence of a test compound. Detection of a greater or lesser amount of fluorescence associated with the lanthanide ion in the presence of the test compound relative to the absence of the test compound identifies the test compound as having the ability to affect expression of the marker protein.

Another embodiment of the invention is a method of detecting a marker protein of a cell. The cell is cultured on a filter plate which has a low background phosphorescence and pores which permit the cell to be cultured on the filter plate and to be washed in situ under vacuum assist. The cell is contacted with a first ligand which specifically binds to the marker protein and washed to remove first ligand which is not specifically bound to the marker protein. The cell is contacted with a reagent comprising a second ligand which specifically binds to the first ligand and a lanthanide chelate. The lanthanide chelate comprises a lanthanide ion and a chelating agent which is bound to the second ligand. The cell is washed to remove reagent which is not specifically bound to the first ligand to provide a washed cell. The washed cell is observed for a sufficient time to detect fluorescence associated with the lanthanide ion which is at least two-fold greater relative to any background fluorescence. Detection of the fluorescence associated with the lanthanide ion indicates the presence of the marker protein. Optionally, the cell is cultured in the presence of a test compound. Detection of a greater or lesser amount of fluorescence associated with the lanthanide ion in the presence of the test compound relative to the absence of the test compound identifies the test compound as having the ability to affect expression of the marker protein.

Still another embodiment of the invention is a test kit which comprises a ligand for a marker protein, a source of lanthanide ion, and a filter plate suitable for culturing cells. The filter plate has a low background phosphorescence and pores which permit cells to be cultured on the filter plate and to be washed in situ.

Even another embodiment of the invention is a test kit comprising a polycarbonate filter plate having an average pore size of about 0.4 μm, a reagent comprising an antibody which specifically binds to a marker protein of an erythroid progenitor cell and a Europium chelate comprising a Europium ion and a chelating agent which is bound to the antibody, a solution comprising a P-diketone and sufficient to dissociate the Europium ion from the chelating agent and instructions for a method of detecting a marker protein of a cell. The cell is cultured on a filter plate which has a low background phosphorescence and pores which permit the cell to be cultured on the filter plate and to be washed in situ. The cell is contacted with a reagent comprising a ligand which specifically binds to the marker protein and a lanthanide chelate. The lanthanide chelate comprises a lanthanide ion and a chelating agent. The chelating agent is bound to the ligand. The cell is washed to remove reagent which is not bound to the marker protein to provide a washed cell. The washed cell is observed for a sufficient time to detect fluorescence associated with the lanthanide ion which is at least two-fold greater relative to any background fluorescence. Detection of the fluorescence associated with the lanthanide ion indicates the presence of the marker protein.

The invention thus provides a rapid and sensitive cell-based assay and test kits for detecting a marker protein of a cell. The assay can be used in a high-throughput format to assess the effect of test compounds on expression of one or more marker proteins of a cell.

### BRIEF DESCRIPTION OF THE FIGURES

FIGS. 1A-1C. Cell surface marker status of erythroid progenitors. Freshly isolated erythroid progenitors were analyzed for the expression of CD36 (FIG. 1A), glycophorin A (FIG. 1B) and the erythropoietin receptor (FIG. 1C) by flow cytometry.

FIG. 2. Differentiation of human erythroid progenitor cells. Cryopreserved progenitors were thawed and cultured in serum-free culture medium containing IL-3, IL-6, EPO, and SCF. Erythroid differentiation was followed by the expression of hemoglobin and cell surface glycophorin A as determined by flow cytometry.

FIG. 3. Comparison of committed human erythroid progenitor cell proliferation in plastic culture plates and filter plates. Cryopreserved progenitors were thawed and seeded into wells of conventional (Coming) 96-well plastic cell culture plates and Millipore MultiScreen PCF filter plates at a density of 10,000 cells/well and cultured in the presence of IL-3, IL-6, EPO, and SCF. Cell densities were determined on days 2, 4, and 6. Each point is based on triplicate determinations from three different experiments.

FIG. 4. Linearity of cellular hemoglobin detection in the filter plate assay. A standard curve was generated by assay of increasing numbers of mature peripheral blood erythrocytes in wells of a 96-well PCF filter plate.

FIG. 5. Adult hemoglobin (HbA) expression by differentiating human erythroid progenitors. Cryopreserved progenitors were thawed and seeded in a T-25 flask at a density of 75,000 cells/ml and cultured in the presence of IL-3, IL-6, EPO and SCF. On days 0, 2, 4, and 6, cells were fixed in 1% paraformaldehyde and subsequently transferred to a PCF filter plate for analysis of hemoglobin content.

FIG. 6. Adult hemoglobin (HbA) expression by differentiating human erythroid progenitors. Cryopreserved progenitors were thawed and seeded into wells of a 96-well PCF filter plate at a density of 1,000 cells/well and cultured in the presence of IL-3, IL-6, EPO, and SCF ±/−0.5 mM succinylacetone. Hemoglobin was assayed by the filter plate procedure on day 7.

FIG. 7. Comparison of committed human hematopoietic CD34⁺ progenitor cell proliferation in plastic culture plates (“control plate”) and Millipore black polycarbonate “MultiScreen Fluorescence” plates (“grey filter plate”).

FIG. 8. Bar graph illustrating use of the CELISA™ method to detect lineage-specific cell-surface marker proteins using a polycarbonate filter plate.

### DETAILED DESCRIPTION OF THE INVENTION

The invention is a new rapid-throughput cell-based assay (“CELISAT™”) and a related test kit that can be used, for example, to screen test compounds for their effect on cell growth and differentiation as indicated by expression of marker proteins of a cell. The components of the method are described in detail below. Briefly, cells are cultured on a filter plate. The filter plate comprises a low background phosphorescence material with pores which permit cells to be cultured on the filter plate and to be washed in situ. Loss of cells can therefore be minimized by using vacuum filtration for wash steps.

Cells on the plate are contacted with a reagent which comprises a ligand which specifically binds to the marker protein and to a lanthanide chelate. The lanthanide chelate comprises a lanthanide ion chelated to a chelating agent, and the chelating agent is bound to the ligand. Cell culture medium can be removed, if desired, before the cells are contacted with the reagent. Optionally, the reagent can be added directly to the cell culture supernatant. Direct addition avoids wash steps and further increases the high throughput nature of the assay.

After washing the cells to remove unbound reagent, fluorescence associated with the lanthanide ion can be measured. The fluorescence associated with the lanthanide ion reflects binding of the ligand to the marker protein and therefore can be used to detect the presence of the marker protein. The high background fluorescence characteristic of cell-based assays can be minimized using time-resolved fluorescence measurement of the fluorescence associated with the lanthanide ion. Various methods, described below, can be used to enhance the fluorescent signal.

In one embodiment of the invention, the ligand is a single primary antibody, which decreases processing time relative to assays which use both primary and secondary antibodies. Alternatively, two ligands, such as a primary and a secondary antibody, or a first ligand coupled to biotin and a second ligand comprising avidin or streptavidin, can be used, as described in more detail below.

The method of the invention can be used, inter alia, to monitor differentiation of a cell, to assess the viability of the cell, or to screen test compounds for various effects on marker protein expression. Because marker protein detection is carried out using the same filter plate on which the cells are cultured, the method is easily adapted for use in high throughput screening formats, thus relieving a significant bottleneck in the screening of test compounds for desired or undesired effects in vivo.

The assay is especially well-suited for screening test compounds for their ability to affect erythropoiesis. This embodiment of the invention takes advantage of the availability of erythroid progenitor cells which are CD36+. These cells require significantly less time in culture than the pluripotent progenitor cells which have previously been used in assays of erythropoiesis. The shortened culture time, together with the sensitivity and other advantages of a reagent comprising a primary antibody conjugated to a lanthanide chelate, dramatically increases the throughput of hematopoietic assays using this invention.

The invention also provides a test kit for use in the screening and detection methods of the invention. The kit comprises a ligand for a marker protein, a source of a lanthanide ion, and a filter plate suitable for culturing cells. The source of the lanthanide ion can be, for example, a lanthanide chelate, a reagent comprising a second ligand bound to a lanthanide chelate, and a reagent comprising a lanthanide chelate and a β-diketone. Each of these sources is described more fully below. The filter plate in the kit has a low background phosphorescence material with pores which permit cells cultured on the filter plate to be washed in situ, also as described more filly below. Optionally, the test kit also includes a cell which is capable of expressing a marker protein. If desired, instructions for various methods of the invention can be provided in the test kit.

### Cells

Cells which can be used in the detection and screening methods of the invention are not limited to any particular type but will generally be mammalian, preferably human, cells. Such cells include cultures of primary cells as well as neoplastic or normal cell lines. Single or multiple cells can be cultured, e.g., maintained in vitro. In one embodiment, the cell is a progenitor cell which is capable of further differentiation, such as a stem cell of the bone, testis, or stratified squamous epithelium, an embryonic stem cell, a neural progenitor cell, a glial progenitor cell, or a hematopoietic progenitor cell, including a granulocytic, monocytic, erythroid, megakaryocytic, myeloid, and lymphoid stem cell. The use of erythroid progenitor cells is specifically contemplated.

Any medium in which a particular cell type can be cultured for expression of the marker protein to be detected can be used in the method of the invention. The selection of an appropriate culture medium for a given cell type, as well as other culture conditions such as temperature and percent CO₂, is well within the skill of those in the art (see, for example, ANIMAL CELL CULTURE, R. I. Freshney, ed., 1986). For example, erythroid progenitor cells preferably are cultured in BioWhittaker HPGM medium, GBSF-58 medium (Quality Biological Inc.), StemSpan SFEM (Stem Cell Technologies), or the equivalent.

### Marker Proteins

Marker proteins which can be detected according to the invention are determined by the nature of the cell being cultured and include both cell-surface and intracellular proteins. The invention is not limited to the use of any particular marker proteins. If an intracellular marker protein is to be detected, cells are preferably fixed and permeabilized, as is known in the art, before incubation with the first ligand or with the ligand-lanthanide chelate reagent. Intracellular marker proteins which can be detected according to the invention include, but are not limited to, hemoglobin A, cytokeratins, cytokines (e.g., IL-4, IFN_{γ}), actin, signal transduction molecules, tartrate-resistant acid phosphatase (a marker for osteoblast differentiation), von Willebrand factor, and neuronal progenitor differentiation markers, such as GFAP, MAP2, beta tubulin III, and nestin.

Marker proteins which can be detected to assess the toxicity of various test compounds are those which are present in many cell types, and include proteins such as aldehyde dehydrogenase (ALDH), collagen, glyceraldehyde 3-phosphate dehydrogenase, elongation factor 1 alpha, and gamma actin. Marker proteins which can be detected to assess the effect of test compounds on the cell cycle include various microtubule and microfilament proteins. Other marker proteins, which are cell type- or cell stage-specific, can be detected to assess the effect of test compounds on cell differentiation. These marker proteins include, for example, hematopoietic markers, such as hemoglobin A, glycophorin A, gpIIbIIIa, the erythropoietin receptor, CD11b, CD19, CD33, CD34, CD36, CD41, MO1, OKT3, OKT4, OKT8, OKT11, OKT16, OKM1, OKM5, Leu7, Leu9, Leu M1, and Leu M3; neuron-specific marker proteins, such as acetylcholinesterase, glial-specific marker proteins, such as glial fibrillary acidic protein (GFAP) and myelin basic protein; and other proteins found only in specialized cells, such as human milk fat globule antigen (HMFG), keratins, and crystallins.

Optionally, two or more marker proteins can be detected in the same cell or cell culture, either simultaneously or sequentially, using ligands which specifically bind to each of the marker proteins. For simultaneous detection, for example, different antibodies can be conjugated to lanthanide chelates having distinguishable excitation and/or emission maxima.

### Reagents Comprising Ligands Bound to Lanthanide Chelates

Typically, a ligand which specifically binds to a marker protein binds to the marker protein with an affinity which is at least about 2- to 3-fold, preferably 5- to 10-fold, even more preferably 20-fold higher than the affinity with which the ligand binds to other proteins when used in an appropriate binding assay. If the ligand is an antibody, for example, appropriate assays include immunochemical assays, such as a Western blot radioimmunoassays, or immunocytochemical assays. Preferably, an antibody which specifically binds to a marker protein does not detect other proteins in immunochemical assays and can immunoprecipitate the marker protein from solution. Binding affinity of a ligand to a receptor, or of a ligand which is a receptor to its own ligand, can be assayed by radioimmunoassay, fluorescence quenching, or other suitable assays known in the art. Antibodies, such as monoclonal or polyclonal antibodies, single-chain antibodies, Fab fragments, (Fab′)2 fragments, and the like, are conveniently used in the method of the invention. The term “antibody” as used in the specification and claims is intended to embrace all of these alternatives.

In one embodiment of the invention, a ligand which specifically binds to the marker protein is bound to a lanthanide chelate. The absorbance of lanthanide chelates is very strong (more than 10⁴fluorescence units). The excitation maximum is within the short UV-range (270-320 nm for terbium-chelates, 320-360 nm for Eu-chelates), which makes it possible to excite them with commercially available lamps or lasers. The Stoke's shift is very long (240-270 nm), and the emission band appears at relatively long wavelengths (e.g., 544 nm for terbium, 613 nm for Europium) and is sharply limited, which permits a narrow band width to be used. The most useful property, however, is that the fluorescence time is long (about 50-1000 microseconds). Thus, if fluorescence is measured with a certain delay during which any background fluorescence has decayed, the effect of an unspecific background radiation can be virtually eliminated.

Preparation of lanthanide chelates which can be conjugated to ligands such as antibodies is taught in U.S. Pat. No. 4,374,120, the disclosure of which is incorporated herein by reference. A lanthanide chelate can be conjugated to an antibody, for example, via an amino polycarboxylic acid analogue, such as an EDTA-analogue, HEDTA-analogue, as taught in U.S. Pat. Nos. 4,374,120, 4,808,541, 5,859,215, 5,457,186, and 5,216,134 all of which are incorporated herein by reference (see also U.S. Pat. Nos. 5,859,215, 5,637,509, 5,571,897, and 4,565,790, also incorporated herein by reference).

In another embodiment of the invention, a first ligand, such as a primary antibody or a biotin-, avidin-, or streptavidin-labeled ligand, is bound to the marker protein. A second ligand conjugated to a lanthanide chelate is then bound to the first ligand. For example, the first ligand can be a primary antibody and the second ligand can be a secondary antibody. Many suitable primary-secondary antibody pairs are well known in the art and can be used in this embodiment of the invention. Alternatively, the first ligand can comprise a biotin moiety and the second ligand can comprise avidin or streptavidin. If desired, the first ligand can comprise avidin or streptavidin and the second ligand can comprise biotin. Other specific binding pairs also can be used, as is known in the art.

### Generation and Detection of Fluorescent Chelates

Fluorescence associated with a lanthanide chelate can be measured without dissociating the lanthanide ion from the chelate (see U.S. Pat. No. 4,808,541) or by using a lanthanide chelate which comprises a β-diketone to amplify the fluorescent signal (see U.S. Pat. No. 4,374,120). Suitable β-diketones are, for example, 2-naphthoyltrifluoroacetone (2-NTA), 1-naphthoyltrifluoroacetone (1-NTA), p-methoxybenzoyltrifluoroacetone (MO-BTA), p-fluorobenzoyltrifluoroacetone (F-BTA), benzoyltrifluoroacetone (BTA), furoyltrifluoroacetone (FTA), naphthoylfuroylmethane (NFM), dithenoylmethane (DTM), and dibenzoylmethane (DBM). Preferably, the lanthanide ion is dissociated from the chelating agent at a low pH-value, typically below 3.5, in a solution which preferably contains a suitable detergent, such as Triton X-100, and a β-diketone to amplify the fluorescence after the separation. To further improve the fluorescence, especially in aqueous solutions, a synergistic compound such as a so-called “Lewis base” can be added. Suitable synergistic compounds include N-heterocyclic compounds (e.g., o-phenanthroline), as well as phosphines and phosphine oxides (e.g. trioctylphosphineoxide) (see U.S. Pat. No. 4,565,790, incorporated herein by reference). The EG&G Wallac DELFIA® method is particularly useful for measuring fluorescence associated with a lanthanide chelate.

One suitable solution comprises 15 μM β-naphthoyltrifluoroacetone, 50 μM trioctylphosphine oxide, 0.1% Triton X-100 in phthalatacetate buffer, pH 3.2 (see U.S. Pat. No. 4,808,541, incorporated herein by reference). DELFIA® Enhancement Solution (EG&G Wallac) is preferred, but any acidic solution in which the lanthanide ion dissociates from the chelating agent and complexes with a second chelating agent, such as a β-diketone, to form a fluorescent chelate can be used. Europium chelates also are preferred, although any other fluorescent lanthanide chelate which generates a fluorescent signal which is at least about two-fold greater, preferably at least about five-fold greater, even more preferably at least about ten-fold greater relative to any background fluorescence from, for example, the cell and any residual culture materials, also can be used.

Detection of the fluorescent chelate can be either qualitative or quantitative. Fluorescence preferably is detected by a method using time delay, which reduces or eliminates the contribution of non-specific background fluorescence to the detected signal. A preferred method of detection is time-resolved fluorometry, which is especially well suited for use with fluorescent lanthanide chelates (14 and U.S. Pat. No. 4,374,120; see also Example 1, below). Devices suitable for carrying out time-resolved fluorimetry include a Victor spectrofluorimeter (e.g. Victor or Victor²™ from EG&G Wallac), SPECTRAmax GEMINI (Molecular Devices), the LJL-Analyst, and FLUOstar from BMG Lab Technologies.

### Filter Plates

Washing steps necessary to remove unbound antibody contribute to the labor-intensive character of immunoassays and decrease their throughput. In addition, loss of cells during washes or during the transfer of a cell culture is always an important concern. In the current assay, this problem has been essentially eliminated by use of porous filter plates on which the cells are cultured and washed in situ. The average pore size of the filter plate is sufficient to permit culture medium, wash fluid, and unbound ligands, etc. to pass through under vacuum assist while retaining the cultured cells. In the absence of such vacuum, the average pore size is such that surface tension effects are sufficient for the filter to act as a culture plate for cells ranging in size, for example, from 5 μm to 30 μm in diameter. Filter plates useful in the inventive methods typically have average pore sizes between 0.001 and 10 μm, preferably between about 0.1 and 5.0 μm, and even more preferably less than about 3.0 μm, or about 0.4 μm.

Thus, the ideal filter plate for use in the inventive methods is cell culture compatible and is constructed, at least in the surface, of a membrane material that has low background phosphorescence and low non-specific protein binding properties. An acceptably low background phosphorescence is preferably about 10,000 counts or less, more preferably about 5000 counts or less, even more preferably about 500 counts or less, as measured, for example, using a spectrofluorimeter (e.g. Victor or Victor²™ from EG&G Wallac, or the equivalent). Additionally, the plates should have an acceptably high vacuum filtration rate, come with both top and bottom covers to minimize evaporation, and be compatible with commercially available vacuum manifolds. Preferably, the plates are available in the well-known 96- or 384-well formats and have a membrane material that is transparent to allow viewing of cells.

Suitable materials for use in filter plates in this invention can be investigated using routine screening techniques. Materials having low phosphorescence, low non-specific protein binding properties, properties which are compatible with cell culture requirements, and which can form pores with average diameters which permit cells to be both cultured in the filter plate and washed in situ, such as polycarbonate, should generally be suitable.

Numerous commercially available filter plates were examined to determine their suitability for use in the assay of this invention. The Millipore MultiScreen PCF filter plate has been determined to exhibit background phosphorescence of approximately 5,000 counts, with excitation at 340 nm and emission at 615 nm. This level of background phosphorescence was 10-fold higher than some other commercially available plates and 20-fold lower than others. However, unlike many commercially available filter plates, the PCF plate also is optimized for cell culture. An even more preferred Millipore filter plate is the black polycarbonate membrane plate designated as the “MultiScreen Fluoresence” plate.

### Test Compounds

Test compounds to be screened for the ability to affect marker protein expression can be any pharmacologic agents already known in the art or can be compounds previously unknown to have any pharmacological activity. Test substances can be naturally occurring or synthesized in the laboratory. They can be isolated from microorganisms, animals, or plants, or can be produced recombinantly or synthesized by chemical methods known in the art.

Because the present invention is conducive to high throughput screening, test compounds typically are obtained from compound libraries. Methods of generating combinatorial libraries of test compounds are known in the art and include, but are not limited to, formation of “biological libraries,” spatially addressable parallel solid phase or solution phase libraries, synthetic library methods requiring deconvolution, the “one-bead one-compound” library method, and synthetic library methods using affinity chromatography selection (15-21). The biological library approach is limited to polypeptide libraries, while the other four approaches are applicable to polypeptide, non-peptide oligomer or small molecule libraries of compounds (13). The present invention, however, is in no way limited to the method for generating, or source of potential test compounds.

Test compounds can be presented to cells, for example, in solution (22), on beads (23), plasmids (24), or phage (25-28 and U.S. Pat. No. 5,223,409, incorporated herein by reference). Optionally, the degree to which the test compound affects expression of the marker protein can be quantitated. For example, fluorescence associated with a lanthanide ion can be quantitated in the presence of the test compound (first fluorescence), and fluorescence associated with the lanthanide ion in the absence of the test compound (second fluorescence) each can be measured. Subtraction of the second fluorescence from the first fluorescence provides the difference between the two fluorescences and, therefore, the degree to which the test compound affects expression of the marker protein. Preferably, the first and second fluorescences are associated with the same type of lanthanide ion (e.g., Europium), so that an effective comparison can be made.

The above disclosure generally describes the present invention, and all patent references cited in this disclosure are incorporated herein by reference. A more complete understanding can be obtained by reference to the following specific examples which are provided for purposes of illustration only and are not intended to limit the scope of the invention. Examples 1-7 describe initial experiments which used Millipore PCF filter plates S23ER061M7 or S2ER061M8. Examples 8-11 use the Millipore black polycarbonate membrane plate designated as the “MultiScreen Fluorescence” plate.

### EXAMPLE 1

### Materials and Methods

Reagents. FITC-conjugated monoclonal antibodies to glycophorin A and CD36, FITC-conjugated isotype controls, FITC-conjugated anti-mouse IgG, and monoclonal antibody to the erythropoietin receptor were purchased from Caltag (Burlingame, Calif.). RPE-labeled anti-mouse IgG was purchased from Southern Biotechnology Associates (Birmingham, Ala.). Monoclonal antibody (CR8006) to human adult hemoglobin was purchased from Cortex Biochem (San Leandro, Calif.). Europium labeled antibody to adult hemoglobin (HbA) and an isotype control were obtained from EG&G Wallac/Akron (Akron, Ohio).

MultiScreen PCF filter plates (catalogue numbers S2ER061M7 [0.4 μm] and S2ER061M8 [3.0 μm]) were obtained from Millipore Corporation (Bedford, Mass.). Ninety-six-well cell culture plates (#25860) were purchased from Corning (Acton, Mass.). Assay buffer, Wash Concentrate and Enhancement Solution were obtained from EG&G Wallac (Turku, Finland). Formaldehyde was purchased from Electron Microscopy Sciences (Washington, Pa.). Triton X-100 was purchased from Boehringer Mannheim (Indianapolis, Id.).

Interleukins 3 and 6 (IL-3 and IL-6), stem cell factor (SCF), and erythropoietin (EPO) were purchased from R&D Systems (Minneapolis, Minn.). MicroBead-conjugated monoclonal antibodies to FITC and CD34 and MiniMacs columns were purchased from Miltenyi Biotec (Auburn, Calif.). Succinylacetone was obtained from Sigma Chemical Co. (St. Louis, Mo.). Ficoll-Paque PLUS was obtained from Amersham Pharmacia Biotech AB (Uppsala, Sweden).

Cells. Umbilical cord blood was obtained from a local hospital after IRB review and approval. Cord blood mononuclear cells were isolated by centrifugation over Ficoll. Cord blood CD34⁺ progenitor cells were isolated by positive immunoselection with anti-CD34 antibody-bound magnetic beads using Miltenyi MiniMacs columns. Purified CD34⁺ cells were greater than 90% pure. Mature erythrocytes from normal donors were obtained by differential centrifugation of peripheral blood.

Flow Cytometry. Cell surface and cytoplasmic antigen expression was analyzed by flow cytometry. Cells were washed in phosphate buffered saline (PBS) containing 5% calf serum (CS) and 0.1% NaN₃ (PBS/CS/NaN₃). For surface antigen detection, cells were incubated with selected FITC-conjugated immunophenotyping antibodies for 30 minutes at 4° C. Cells were then washed twice in PBS/CS/NaN₃ and analyzed.

Detection of Hemoglobin A. For detection of hemoglobin A, 10⁶ cells/ml were fixed by incubation in 1% paraformaldehyde at room temperature for 20 minutes. Fixed cells were washed three times in PBS/CS/NaN₃, resuspended at 10⁶ cells/ml, and were stored at 4° C. in the dark until stained. For staining, 2-5×10⁵ fixed cells were permeabilized by incubation in 0.1% Triton X-100 in PB S/CS/NaN₃ for 10 minutes at room temperature. Permeabilized cells were washed twice in PBS/CS/NaN₃ and incubated with anti-Hemoglobin A for 30 minutes at 4° C. Cells were washed twice in PBS/CS/NaN₃ and incubated with FITC-conjugated goat anti-mouse IgGl for 15 minutes at 4° C. Stained cells were washed twice in PBS/CS/NaN₃ and analyzed. Flow cytometry was performed using a FACSort flow cytometer and Lysis II software (Becton Dickinson).

### EXAMPLE 2

### Isolation and Characterization of CD36⁺ Erythroid Progenitors

The integral membrane glycoprotein CD36 was originally isolated from the apical membranes of terminally differentiated mammary secretory epithelial cells and was subsequently found on the surface of platelets, endothelial cells, adipocytes, and other cell types (3). CD36 was found to be a marker of early erythroid development by Kieffer et al. (4) and was used to isolate erythroid progenitor cells by Fichelson et al. (5).

In this example, human erythroid progenitor cells were derived from umbilical cord blood CD34⁺ progenitors as follows. Cryopreserved CD34⁺ progenitor cells were placed in culture in serum-substituted IMDM culture medium containing penicillin, streptomycin, and the growth factors IL-3 (10 ng/ml), IL-6 (10 ng/ml), and SCF (25 ng/ml).

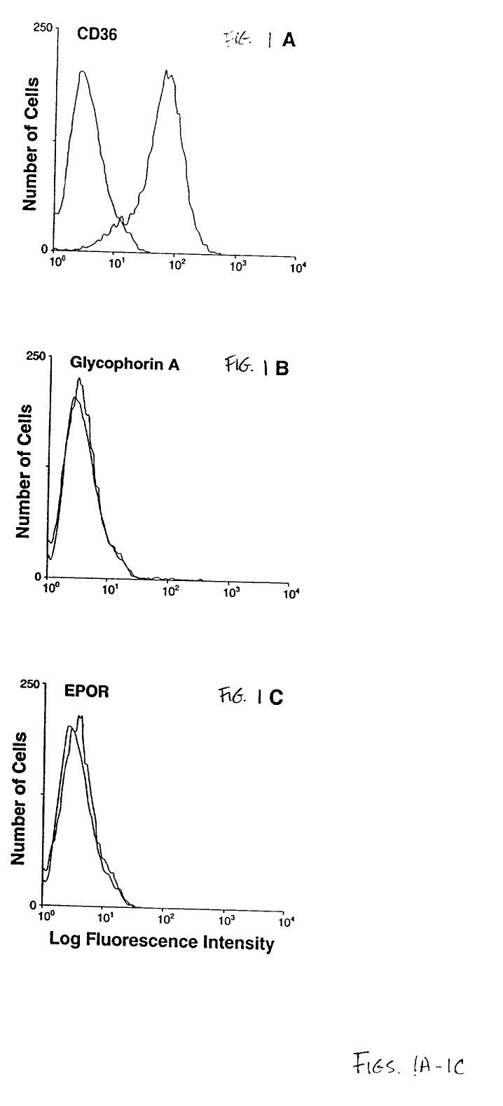

After 1 week, during which time the cells expanded 20- to 30-fold, the cells were washed, incubated with FITC-labeled monoclonal to CD36, rinsed, and incubated with magnetic beads conjugated with antibody to FITC. The CD36⁺ fraction of these cells was isolated by positive immunoselection in a MiniMacs column and cryopreserved. Human erythroid progenitors isolated from umbilical cord blood CD34⁺ progenitors were >90% CD36⁺ (FIG.1A).

The CD36⁺ fraction increased from less than 1% to approximately 30% of the total cell population during the seven days of culture with IL-3, IL-6, and SCF. The CD36⁺ erythroid progenitor cells did not express cell surface markers characteristic of the early stages of erythrocyte differentiation, such as glycophorin A and the erythropoietin receptor (FIG. 1B and FIG. 1C).

### EXAMPLE 3

### Differentiation of CD36⁺ Erythroid Progenitors

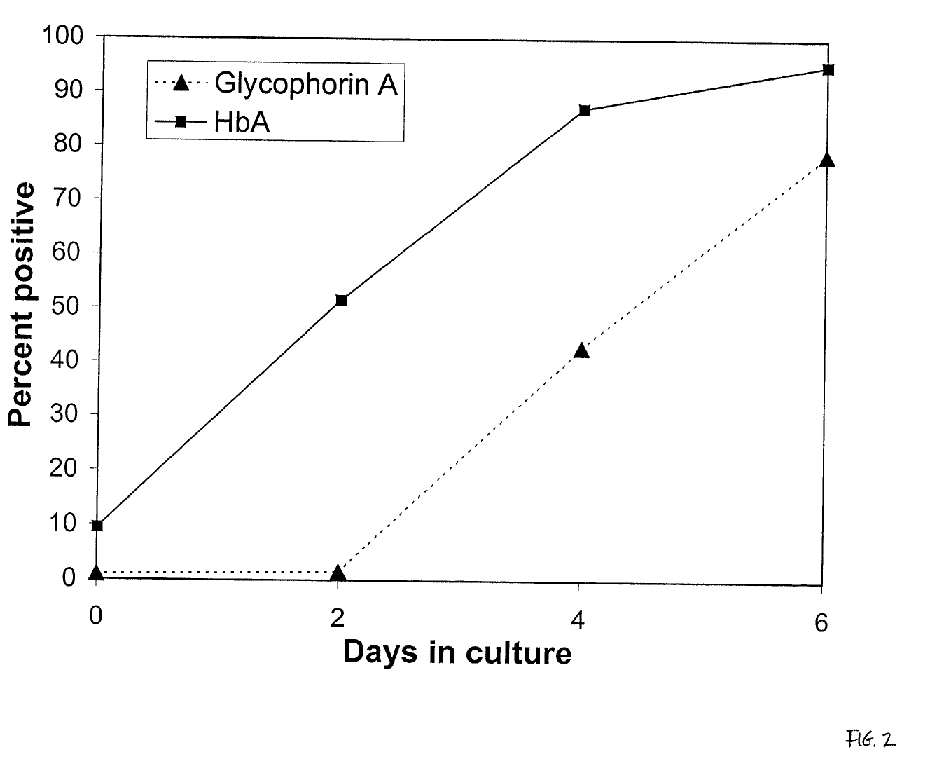

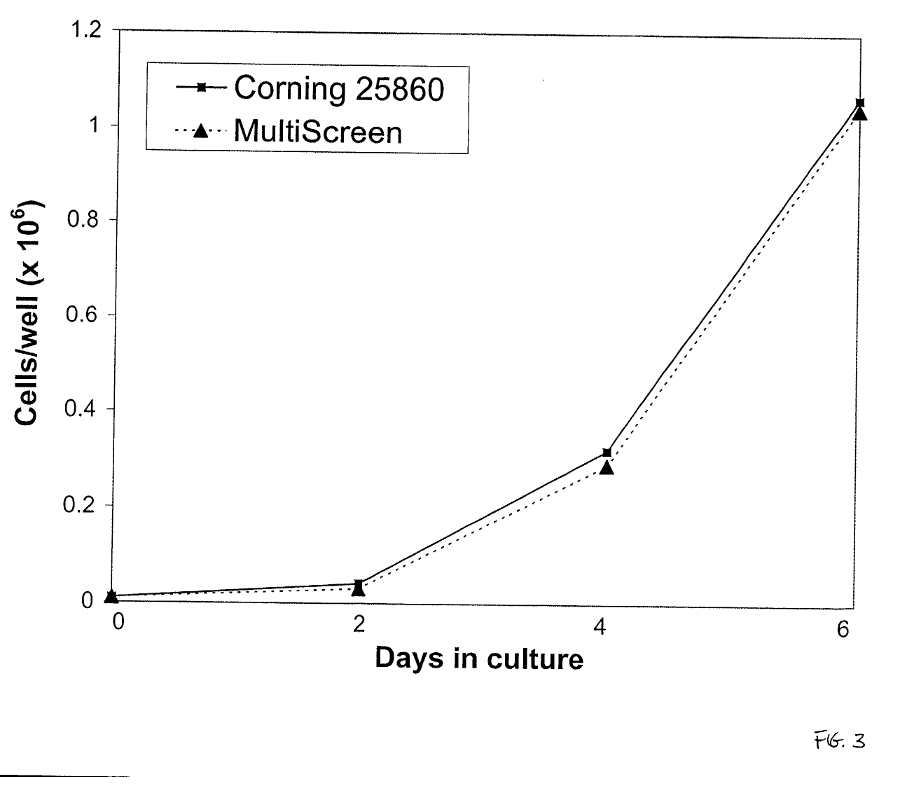

Erythroid progenitors were cultured in serum-free IMDM culture medium containing IL-3 (10 ng/ml), IL-6 (10 ng/ml), SCF (25 ng/ml), and EPO (3 U/ml). Hemoglobin was detected in cells soon after initiation of the culture (FIG. 2). Expression of the erythroid-specific cell surface protein glycophorin A was detected on day 4 cells and continued to increase over the period of the culture. An expansion in cell number of approximately 100-fold was routinely observed over a period of 7 days in the presence of EPO (FIG. 3) in both standard cell culture plates and in filter plates suitably used in the present invention.

Previous studies of the in vitro differentiation of erythroid cells have utilized two-phase culture systems that involved an initial 7-9 day culture period in either semisolid or liquid medium containing serum followed by a second culture period of 5-25 days in serum-containing medium (6, 7). The ability to thaw human erythroid progenitor cells and culture erythroid cells in various stages of terminal differentiation within days provides the opportunity to increase the throughput of toxicity and drug discovery assays with this particular cell lineage.

### EXAMPLE 4

### Proliferation of CD36⁺ Erythroid Progenitors on Filter Plates

Although the PCF filter plate is advertised as optimized for cell culture, it was important to demonstrate that erythroid progenitors proliferated normally in these plates relative to conventional plastic cell culture plates. To do this, erythroid progenitors were thawed and seeded at 10,000 cells/well in both conventional cell culture plates (Coming) and Millipore MultiScreen PCF filter plates. Each set of cells was cultured in the presence of IL-3, IL-6, SCF, and EPO. As shown in FIG. 3, the human erythroid progenitors proliferated at nearly identical rates in both types of plates.

### EXAMPLE 5

### Filter Plate Assay

Hemoglobin was selected as a marker of erythroid differentiation. Hemoglobin is a well-characterized product of the terminally differentiated erythrocyte, and the temporal parameters of its expression relative to other markers of erythrocyte maturation are well known (8, 9). Europium-conjugated monoclonal antibody to adult hemoglobin (HbA) was used to obtain the sensitivity required without use of a secondary antibody for signal amplification. The ability of the assay of this invention to avoid the need for an incubation period with a secondary antibody and the associated washes greatly decreased the time required for the assay. In addition, the relatively long emission time of the Europium-conjugated antibody allowed use of time-resolved fluorimetry to avoid endogenous background fluorescence that is often a significant problem in cell-based assays (10).

To establish the linearity of the assay relative to cell number, peripheral blood erythrocytes were seeded into wells of PCF filter plates (0.4 μm pore size for peripheral blood erythrocytes and 3.0 μm pore size for progenitor cells), fixed with 0.1 ml 1% formaldehyde for 30 minutes and then washed once with Hank's balanced salt solution (HBSS) (0.2 ml/well) and twice with HBSS containing 0.1% bovine serum albumin (BSA). All washes were done in a Millipore Silent Screen vacuum manifold with a vacuum pressure of 5″ Hg.

The cells were then permeabilized by incubation with 0.2 ml 0.1% Triton X-100 in HBSS-BSA for 10 minutes at room temperature. The wells were then rinsed once with 0.2 ml HBSS-BSA and incubated with 0.2 ml Europium-labeled monoclonal antibody to HbA or with an isotype control (1 μg/ml). After 45 minutes at room temperature, the wells were rinsed 3 times with Wash Buffer (Wallac). Enhancement Solution (Wallac) was added (0.1 ml/well), and after 5 minutes Europium fluorescence (excitation at 340 μm and emission at 615 nm) was measured over a 400 μsecond time period after an initial delay of 400 μseconds with a Wallac Victor spectrofluorimeter.

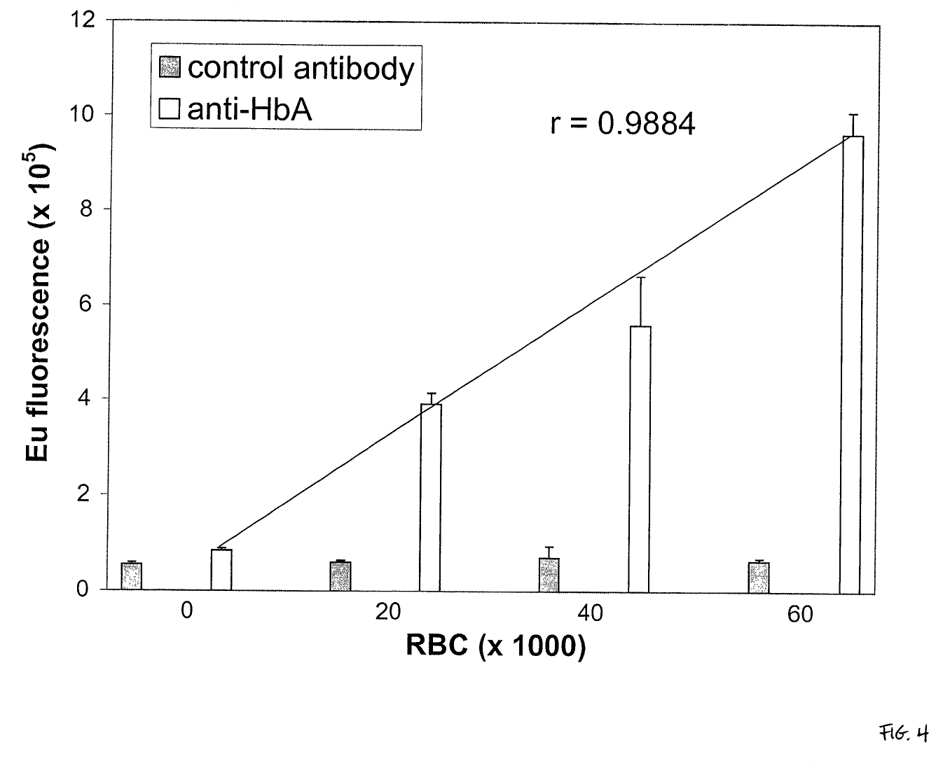

The assay was linear over the working range expected for the erythropoietic assay (0-60,000 cells), with a correlation coefficient of 0.988 and a lower detection limit of approximately 5,000 cells (FIG. 4). The ratio of the fluorescent signal to background fluorescence was approximately 15 for wells containing 60,000 erythrocytes.

### EXAMPLE 6

### Detection of Hemoglobin on a Per Cell Basis

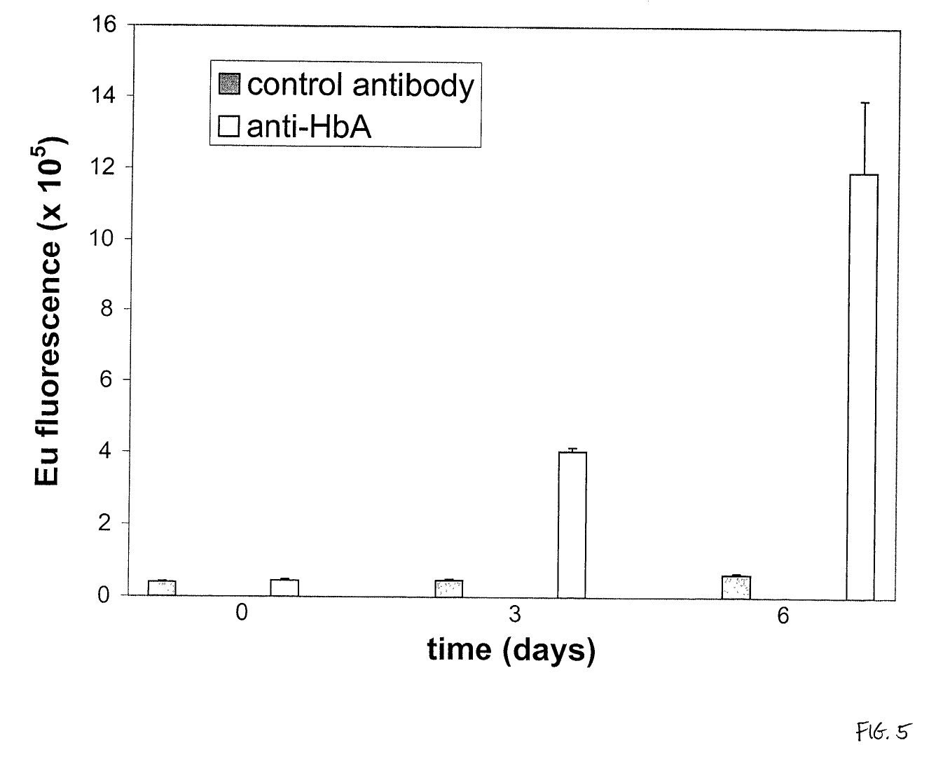

Because increased levels of hemoglobin in the erythroid culture result from both increased hemoglobin/cell and increased cell numbers, an experiment was done to demonstrate that the assay could detect increased hemoglobin on a per cell basis during the differentiation of the erythroid progenitor (FIG. 5). Erythroid progenitors were cultured in a T-25 flask and fixed in 1% formaldehyde on days 0, 3, and 6. Cells (50,000/well) were placed in the wells of a 3.0 μm PCF plate, permeabilized, and assayed for hemoglobin content in situ as described above. The experiment documented a time-dependent increase in the amount of cellular hemoglobin in the differentiating cells, with a signal to noise ratio of 20 in the assay of 6-day cells. The lack of HbA expression in day 0 cells determined in this assay differed from the flow cytometry results, which showed low levels of HbA-expressing day 0 cells (FIG. 2). This variation may be due to the fact that cells used in the filter plate assay and in the flow cytometry assay were obtained from different donors.

### EXAMPLE 7

### Detection of the Effect of an Inhibitor of Hemoglobin Synthesis

To examine the effect of a known inhibitor of hemoglobin synthesis, thawed erythroid progenitors (1,000/well) were cultured in a 3.0 μm pore size PCF filter plate in a cocktail of IL-3, IL-6, SCF, and EPO. Some wells were treated with 0.5 mM succinylacetone, an inhibitor of heme biosynthesis (11). After 7 days, the cells were fixed, permeabilized, and incubated with Europium-labeled anti-hemoglobin or isotype control in situ as described above.

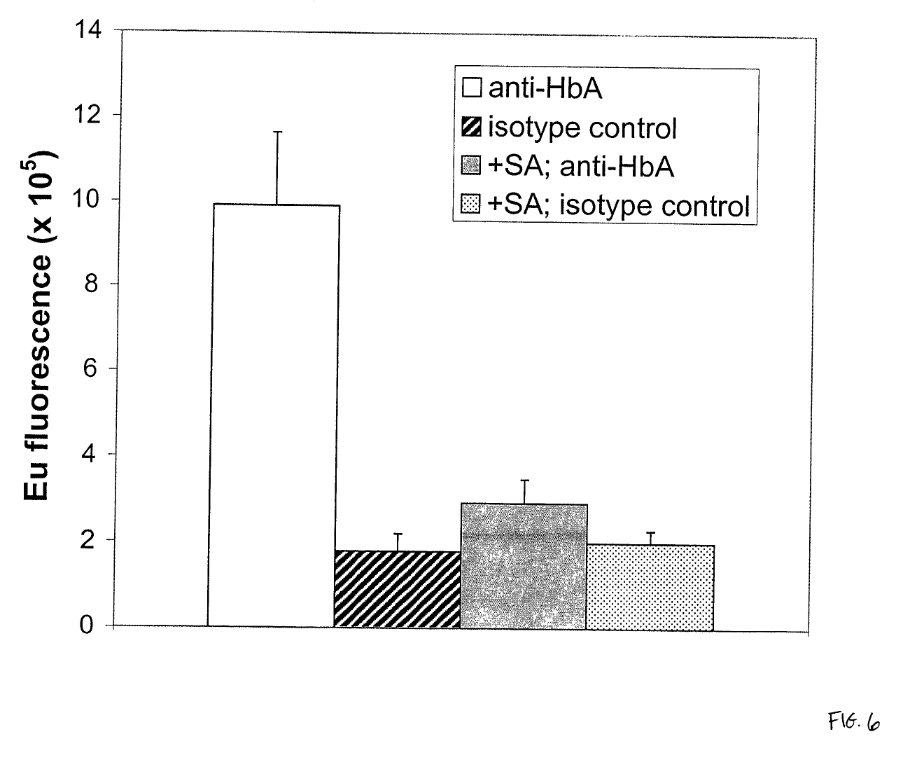

The signal/background ratio of the fluorescent signal in the absence of succinylacetone was 5.5 (FIG. 6). This ratio was significantly lower than that observed for assays of purified erythrocytes (15×) and pre-fixed progenitor cells after 6 days of differentiation (20×). The lower signal to background ratio resulted from significantly higher nonspecific binding in wells in which cells were grown (isotype control signals were approximately 2- to 3-fold higher than those observed in the assays with the purified erythrocytes and pre-fixed progenitor cells). Non-specific background is significantly reduced using polycarbonate filter plates (see Examples 8-10).

The assay effectively detected the inhibition of erythroid differentiation-induced hemoglobin synthesis mediated by succinylacetone, an inhibitor of the second enzyme in the pathway of heme biosynthesis, δ-aminolevulonic acid dehydrogenase (11). The succinylacetone-mediated inhibition of hemoglobin observed in this assay was 86%, a value nearly identical to that observed in other in vitro measurements of this drug's inhibition of hemoglobin biosynthesis (12).

Thus, the filter-plate assay of this invention is useful for detecting the effect of compounds which affect differentiation of a progenitor cell.

### EXAMPLE 8

### Comparison of Committed Human Hematopoietic CD34⁺ Progenitor cell proliferation in Plastic Culture Plates and Millipore Black Polycarbonate “MultiScreen Fluorescence” Plates

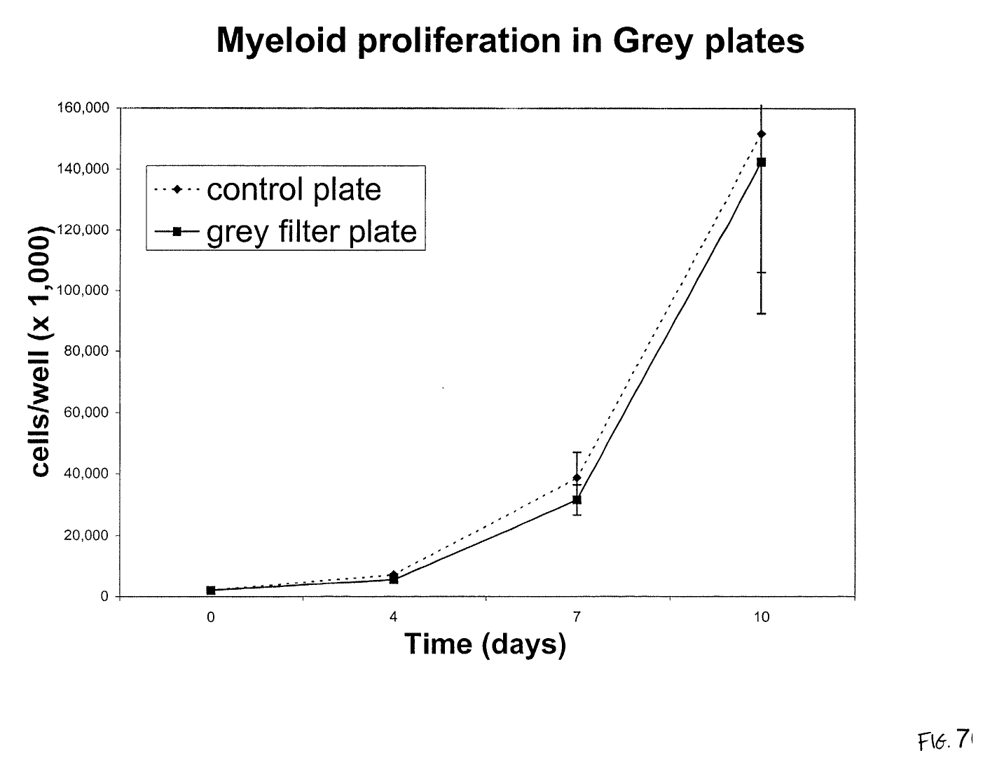

As with erythroid progenitor proliferation in the PCF plates, it was essential to determine that the preferred black polycarbonate plates also were compatible with cell culture. Cryopreserved progenitors were thawed and seeded into wells of conventional 96-well plastic cell culture plates (Coming) and Millipore black polycarbonate “MultiScreen Fluorescence” plates at a density of 2,000 cells/well and cultured in the presence of SCF, G-CSF, GM-CSF, and 15% FBS. Cell densities were determined on days 4, 7, and 10. As with the earlier study of erythroid progenitor proliferation in the PCF filter plates, the preferred black polycarbonate plates alowed for the proliferation of hematopoietic cells during in vitro differentiation and exhibited growth kinetics nearly identical to plastic cell culture plates (FIG. 7).

### EXAMPLE 9

### Quantification of Myeloid Lineage Differentiation with an Antibody to a Lineage-specific Cell Surface Protein

Mature blood cells of the myeloid lineage, such as monocytes and neutrophils, express the integrin subunit CD11b on their surface. The differentiation of these cells from bone marrow-derived progenitor cells can be detected and quantified by measurement of the CD11b protein. This assay can be done in a high-throughput format through use of polycarbonate filter plates to culture the cells and Europium-labeled antibody to CD11b to detect the differentiated cells.

Human bone marrow CD34′ progenitor cells were purified by positive immunomagnetic selection and seeded into the wells of a sterilized Millipore black polycarbonate membrane plate designated as the “MultiScreen Fluorescence” plate (0.4 μm pore size) at 2,000 cells/2001 μl/well in BioWhittaker HPGM medium. Cells were cultured on the plate at 37° C. in 5% CO₂ for 10 days.

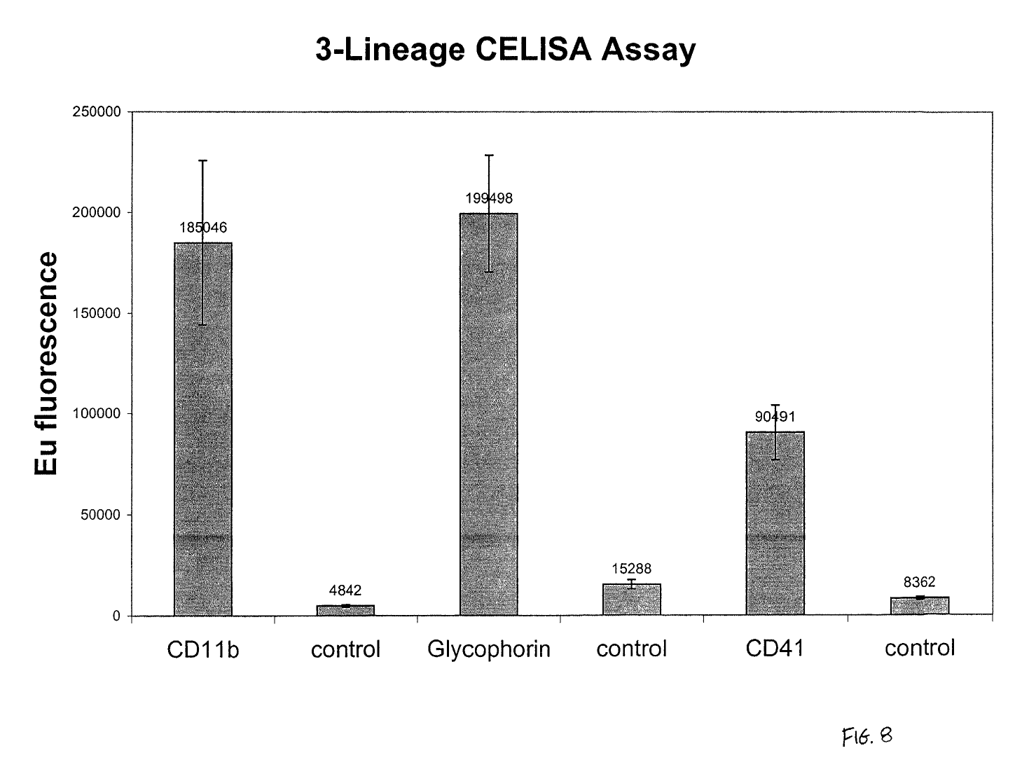

After 10 days, the filter plate was removed from the incubator and placed on a vacuum manifold previously adjusted to provide a vacuum pressure of approximately 5″ Hg. The cell culture medium was removed by vacuum filtration and 200 μl of Tris-buffered saline, pH 7.5 was placed in each well. The Tris saline wash was removed by vacuum filtration, and this wash step was repeated a second time. Each well of the filter plate was then filled with either 200 μl of Europium-labeled monoclonal antibody to the cell surface protein CD11b at a final concentration of 1 μg/ml or an irrelevant isotype control monoclonal antibody at the same concentration. Both antibodies were diluted in Wallac Assay Buffer. The filter plate was then incubated for 60 minutes at room temperature after which the unbound antibody was removed by vacuum filtration. The wells were then rinsed three times with a sequential series of 200 μl, 230 μl and 250 μl Wallac Wash Buffer adjusted so as to contain an additional 0.5 M NaCl. Enhancement Solution (Wallac; 100 μl) was added to each well and, after 5 minutes, Europium fluorescence (excitation at 340 nm and emission at 615 nm) was measured over a 400 μsecond time period after an initial delay of 400 μseconds with a Wallac Victor spectrofluorimeter. The results (FIG. 8) demonstrate a signal to noise ratio of 38.

### EXAMPLES 10

### Quantification of Erythroid Lineage Differentiation with an Antibody to a Lineage-Specific Cell Surface Protein

Erythrocytes, mature blood cells of the erythroid lineage, express the mucin glycophorin A on their surface. The differentiation of these cells from bone marrow-derived progenitor cells can be detected and quantified by measurement of the glycophorin A protein. This assay can be done in a high-throughput format through use of porous polycarbonate filter plates to culture the cells and Europium-labeled antibody to glycophorin A to detect the differentiated cells.

Human bone marrow CD34′ progenitor cells were purified by positive immunomagnetic selection and seeded into the wells of a sterilized Millipore black polycarbonate membrane plate designated as the “MultiScreen Fluorescence” plate (0.4 μm pore size) at 2,000 cells/2001 μ/well in BioWhittaker HPGM medium. Cells were cultured on the plate at 37° C. in 5% CO₂ for 10 days.

After 10 days, the filter plate was removed from the incubator and placed on a vacuum manifold previously adjusted to provide a vacuum pressure of approximately 5″ Hg. The cell culture medium was removed by vacuum filtration and 200 μl of Tris-buffered saline, pH 7.5 was placed in each well. The Tris saline wash was removed by vacuum filtration, and this wash step was repeated a second time. Each well of the filter plate was then filled with either 200 μl of Europium-labeled monoclonal antibody to the cell surface protein glycophorin A at a final concentration of 2 μg/ml or an irrelevant isotype control monoclonal antibody at the same concentration. Both antibodies were diluted in Wallac Assay Buffer. The filter plate was then incubated for 60 minutes at room temperature, after which the unbound antibody was removed by vacuum filtration. The wells were then rinsed three times with a sequential series of 200 μl, 230 μl and 250 μl Wallac Wash Buffer adjusted so as to contain an additional 0.5 M NaCl. Enhancement Solution (Wallac; 100 μl) was added to each well and, after 5 minutes, Europium fluorescence (excitation at 340 nm and emission at 615 μm) was measured over a 400 μsecond time period after an initial delay of 400 μseconds with a Wallac Victor spectrofluorimeter. The results (FIG. 8) demonstrate a signal to noise ratio of 13.

### EXAMPLE 11

### Quantification of Megakaryocytic Lineage Differentiation with an Antibody to a lineage-Specific Cell Surface Protein

Mature blood cells of the megakaryocytic lineage express the integrin subunit CD41a on their surface. The differentiation of these cells from bone marrow-derived progenitor cells can be detected and quantified by measurement of the CD41a protein. This assay can be done in a high-throughput format through use of porous polycarbonate filter plates to culture the cells and Europium-labeled antibody to CD41a to detect the differentiated cells.

Human bone marrow CD34⁺ progenitor cells were purified by positive immunomagnetic selection and seeded into the wells of a sterilized Millipore black polycarbonate membrane plate designated as the “MultiScreen Fluorescence” plate (0.4 μm pore size) at 2,000 cells/200 μl/well in BioWhittaker HPGM medium. Cells were cultured on the plate at 37° C. in 5% CO₂ for 10 days.

After 10 days, the filter plate was removed from the incubator and placed on a vacuum manifold previously adjusted to provide a vacuum pressure of approximately 5″ Hg. The cell culture medium was removed by vacuum filtration and 200 μl of Tris-buffered saline, pH 7.5 was placed in each well. The Tris saline wash was removed by vacuum filtration, and this wash step was repeated a second time. Each well of the filter plate was then filled with either 200 μl of Europium-labeled monoclonal antibody to the cell surface protein CD41a at a final concentration of 1 μg/ml or an irrelevant isotype control monoclonal antibody at the same concentration. Both antibodies were diluted in Wallac Assay Buffer. The filter plate was then incubated for 60 minutes at room temperature after which the unbound antibody was removed by vacuum filtration. The wells were then rinsed three times with a sequential series of 200 μl, 230 μl and 250 μl Wallac Wash Buffer adjusted so as to contain an additional 0.5 M NaCl. Enhancement Solution (Wallac; 100 μl) was added to each well and, after 5 minutes, Europium fluorescence (excitation at 340 nm and emission at 615 nm) was measured over a 400 μsecond time period after an initial delay of 400 μseconds with a Wallac Victor spectrofluorimeter. The results (FIG. 8) demonstrate a signal to noise ratio of greater than 10.

It will be understood that while the invention has been described in conjunction with specific embodiments thereof, the foregoing description and examples are intended to illustrate, but not limit the scope of the invention. Other aspects, advantages and modifications will be apparent to those skilled in the art to which the invention pertains, and these aspects and modifications are within the scope of the invention, which is limited only by the appended claims.

### REFERENCES

1. Eaves, C. J. (1995) Assays of hematopoietic progenitor cells. In WILLIAMS HEMATOLOGY, 5. McGraw-Hill, Beutler et al., eds., pp. L22-6.

2. Warren et al. (1995) CD34⁺ cell expansion and expression of lineage markers during liquid culture of human progenitor cells. Stem Cells (Dayt) 13:167.

3. Greenwalt et al. (1992) Membrane glycoprotein CD36: a review of its roles in adherence, signal transduction, and transfusion medicine. Blood80(5):1105.

4. Kieffer et al. (1989) Developmentally regulated expression of a 78 kDa erythroblast membrane glycoprotein immunologically related to the platelet thrombospondin receptor. Biochem J. 262:835.

5. Fichelson et al. (1998) Purification of a population of human BFU-E from CD34⁺ cord blood cell cultures. Exp. Hematology 26:763A.

6. Fibach et al. (1991) Growth of normal erythroid progenitors in liquid culture: A comparison with colony growth in semisolid culture. Int. J. Cell Cloning 9:57.

7. Panzenbock et al. (1998) Growth and differentiation of human stem cell factor/erythropoietin-dependent erythroid progenitor cells in vitro. Blood 92:3658.

8. Nakahata et al. (1994) Cell surface antigen expression in human erythroid progenitors: erythroid and megakaryocytic markers. Leuk Lymphoma 13:401-409.

9. Okumura et al. (1992) Changes in cell surface antigen expressions during proliferation and differentiation of human erythroid progenitors. Blood 80(3):642.

10. Barnard et al. (1988) Time-resolved fluoroimmunoassay. In COMPLEMENTARY IMMUNOASSAYS, John Wiley and Sons, Chichester, UK. (W. P. Collins, ed.), pp. 149-167.

11. Sassa & Kappas (1983) Hereditary tyrosinemia and the heme biosynthetic pathway: Profound inhibition of the delta-aminolevulinic acid dehydratase activity by succinylacetone. J. Clin. Invest. 71: 625.

12. Fibach et al. (1995) Hemin-induced acceleration of hemoglobin production in immature cultured erythroid cells: Preferential enhancement of fetal hemoglobin. Blood 85:2967.

13. Lam (1997) Anticancer Drug Des. 12, 145.

14. Soini et al. (1979) Clin. Chem. 25, 353-61.

15. DeWitt et al. (1993) Proc. Natl,'Acad. Sci. U.S.A. 90, 6909.

16. Erb et al. (1994) Proc. Natl. Acad. Sci. U.S.A. 91, 11422.

17. Zuckermann et al. (1994) J. Med. Chem. 37, 2678.

18. Cho et al. (1993) Science 261, 1303.

19. Carell et al. (1994) Angew. Chem. Int. Ed. Engl. 533, 2059.

20. Carell et al. (1994) Angew. Chem. Int. Ed. Engl. 33, 2061.

21. Gallop et al. (1994) J. Med. Chem. 37,1233.

22. Houghten (1992) Biotechniques 13, 412-421.

23. Lam (1991) Nature 354, 82-84.

24. Cull et al. (1992) Proc. Natl. Acad. Sci. U.S.A. 89, 1865-1869.

25. Scott & Smith (1990) Science 249, 386-390.

26. Devlin (1990) Science 249, 404-406.

27. Cwirla et al. (1990) Proc. Natl. Acad. Sci. 97, 6378-6382

28. Felici (1991)J. Mol. Biol. 222, 301-310.

1. A method of detecting a marker protein of a cell, comprising the steps of:
(a) culturing the cell on a filter plate, wherein the filter plate has:
(i) a low background phosphorescence; and
(ii) pores which permit the cell to be cultured on the filter plate and to be washed in situ;
(b) contacting the cell with a reagent comprising:
(i) a ligand which specifically binds to the marker protein; and
(ii) a lanthanide chelate comprising a lanthanide ion and a chelating agent, wherein the chelating agent is bound to the ligand;
(c) washing the cell with vacuum assist to remove reagent which is not bound to the marker protein to provide a washed cell; and
(d) observing the washed cell for a sufficient time to detect fluorescence associated with the lanthanide ion which is at least two-fold greater relative to any background fluorescence, wherein detection of the fluorescence associated with the lanthanide ion indicates the presence of the marker protein.

2. The method of claim 1 wherein the reagent further comprises a β-diketone.

3. The method of claim 1 further comprising the step of contacting the washed cell with a solution sufficient to dissociate the lanthanide ion from the chelating agent, wherein the solution comprises a β-diketone.

4. The method of claim 1 wherein the fluorescence associated with the lanthanide ion is at least about five-fold greater.

5. The method of claim 1 wherein the fluorescence associated with the lanthanide ion is at least about ten-fold greater.

6. The method of claim 1 wherein the marker protein is a cell-specific marker protein.

7. The method of claim 1 wherein the marker protein is a cell-surface protein.

8. The method of claim 7 wherein the cell surface protein is selected from the group consisting of glycophorin A, CD11b, CD19, CD34, CD36, CD41, gpIIbIIIa, and an erythropoietin receptor.

9. The method of claim 1 wherein the marker protein is an intracellular protein.

10. The method of claim 9 wherein the intracellular protein is selected from the group consisting of hemoglobin A, a cytokine, a cytokeratin, actin, a signal transduction molecule, tartrate-resistant acid phosphatase, von Willebrand factor, GFAP, MAP2, beta tubulin III, and nestin.

11. The method of claim 1 wherein the cell is contacted with at least two ligands, wherein each ligand specifically binds to a different marker protein of the cell.

12. The method of claim 1 wherein the lanthanide chelate is a Europium chelate.

13. The method of claim 1 wherein the pores of the filter plate are 3.0 82 m or below in average diameter.

14. The method of claim 13 wherein the pores of the filter plate are at least about 0.4 μm in average diameter.

15. The method of claim 1 wherein the filter plate comprises at least 96 discrete wells.

16. The method of claim 1 wherein the filter plate comprises at least 384 discrete wells.

17. The method of claim 1 wherein the cell is a progenitor cell.

18. The method of claim 17 wherein the progenitor cell is selected from the group consisting of an embryonic stem cell, an erythroid progenitor cell, and a neural progenitor cell.

19. The method of claim 1 wherein the filter plate is a polycarbonate filter plate.

20. The method of claim 1 wherein the fluorescence associated with the lanthanide chelate is detected by time-resolved fluorimetry.

21. The method of claim 1 wherein the cell is cultured in the presence of a test compound, and wherein detection of a greater or lesser amount of fluorescence associated with the lanthanide ion in the presence of the test compound relative to the absence of the test compound identifies the test compound as having the ability to affect expression of the marker protein.

22. The method of claim 21, further comprising the steps of:
measuring a first fluorescence associated with a lanthanide ion in the presence of the test compound;
measuring a second fluorescence associated with the lanthanide ion in the absence of the test compound; and
subtracting the second fluorescence from the first fluorescence to obtain the difference between the fluorescences.

23. A method of detecting a marker protein of a cell, comprising the steps of:
(a) culturing the cell on a filter plate, wherein the filter plate has:
(i) a low background phosphorescence; and
(ii) pores which permit the cell to be cultured on the filter plate and to be washed in situ;
(b) contacting the cell with a first ligand which specifically binds to the marker protein;
(c) washing the cell with vacuum assist to remove first ligand which is not specifically bound to the marker protein;
(d) contacting the cell with a reagent comprising:
(i) a second ligand which specifically binds to the first ligand; and
(ii) a lanthanide chelate comprising a lanthanide ion and a chelating agent, wherein the chelating agent is bound to the second ligand;
(e) washing the cell with vacuum assist to remove reagent which is not specifically bound to the first ligand to provide a washed cell; and
(f) observing the washed cell for a sufficient time to detect fluorescence associated with the lanthanide ion which is at least two-fold greater relative to any background fluorescence, wherein detection of the fluorescence associated with the lanthanide ion indicates the presence of the marker protein.

24. The method of claim 23 wherein either:
(1) the first ligand comprises a biotin moiety and the second ligand comprises a moiety selected from the group consisting of avidin and streptavidin;
(2) the first ligand comprises a moiety selected from the group consisting of avidin and streptavidin and the second ligand comprises a biotin moiety; or
(3) the first ligand is a primary antibody and the second ligand is a secondary antibody.

25. The method of claim 23 wherein the cell is cultured in the presence of a test compound, and wherein detection of a greater or lesser amount of fluorescence associated with the lanthanide ion in the presence of the test compound relative to the absence of the test compound identifies the test compound as having the ability to affect expression of the marker protein.

26. The method of claim 23, further comprising the steps of:
measuring a first fluorescence associated with a lanthanide ion in the presence of the test compound;
measuring a second fluorescence associated with the lanthanide ion in the absence of the test compound; and
subtracting the second fluorescence from the first fluorescence to obtain the difference between the fluorescences.

27. A test kit, comprising:
(a) a ligand for a marker protein;
(b) a source of lanthanide ion; and
(c) a filter plate suitable for culturing cells, having:
(1) a low background phosphorescence; and
(2) pores which permit cells to be cultured on the filter plate and to be washed in situ.

28. The test kit of claim 27 further comprising instructions for the method of claim 1.

29. The test kit of claim 27 further comprising instructions for the method of claim 23.

30. The test kit of claim 27 further comprising a cell which can express the marker protein.

31. The test kit of claim 30, wherein the cell is a progenitor cell.

32. The test kit of claim 31, wherein the progenitor cell is selected from the group consisting of an embryonic stem cell, an erythroid progenitor cell, and a neural progenitor cell.

33. The test kit of claim 27, wherein the lanthanide is Europium.

34. The test kit of claim 27 wherein the filter plate comprises at least 96 discrete wells.

35. The test kit of claim 34 wherein the filter plate comprises at least 384 discrete wells.

36. The test kit of claim 27 wherein the marker protein is a cell-specific marker protein.

37. The test kit of claim 36 wherein the marker protein is a cell-surface marker protein.

38. The test kit of claim 37 wherein the cell-surface marker protein is selected from the group consisting of glycophorin A, CD11b, CD19, CD34, CD36, CD41, gpIIbIIIa, and an erythropoietin receptor.

39. The test kit of claim 36 wherein the marker protein is an intracellular protein.

40. The test kit of claim 39 wherein the intracellular protein is selected from the group consisting of hemoglobin A, a cytokine, a cytokeratin, actin, a signal transduction molecule, tartrate-resistant acid phosphatase, von Willebrand factor, GFAP, MAP2, beta tubulin III, and nestin.

41. The test kit of claim 27 wherein the source of lanthanide ion is selected from the group consisting of a lanthanide chelate, a reagent comprising a ligand bound to a lanthanide chelate, and a reagent comprising a lanthanide chelate and a β-diketone.

42. A test kit, comprising:
(a) a polycarbonate filter plate having an average pore size of about 00.4 μm;
(b) a reagent comprising:
(i) an antibody which specifically binds to a marker protein of an erythroid progenitor cell; and
(ii) a Europium chelate comprising a Europium ion and a chelating agent, wherein the chelating agent is bound to the antibody;
(c) a solution comprising a β-diketone and sufficient to dissociate the Europium ion from the chelating agent; and
(d) instructions for the method of claim 1.

43. The test kit of claim 42 further comprising a CD36⁺ erythroid progenitor cell.
